(19) United States
(12) Patent Application Publication
(10) Pub. No.: US 2009/0175945 A1
(43) Pub. Date: Jul. 9, 2009

(54) Systems, Methods, and Formulations for Topically Treating Nail Fungal Infections and Nail Psoriasis

(76) Inventors: Jie Zhang, Salt Lake City, UT (US); Kevin S. Warner, West Jordan, UT (US); Promod Kumar, Mason, OH (US); Hyder Aliyar, Salt Lake City, UT (US)

Correspondence Address: THORPE NORTH & WESTERN, LLP., P.O. Box 1219, SANDY, UT, 84091-1219, US

(21) Appl. No.: 12/349,922

(22) Filed: Jan. 7, 2009

Related U.S. Application Data
(60) Provisional application No. 61/010,500, filed on Jan. 8, 2008.

(51) Int. Cl. A61K 9/10; A61K 31/135; A61K 9/00; A61P 31/10

(52) U.S. Cl. 424/484; 514/649; 424/400

(57) ABSTRACT

The present invention is drawn to systems, methods, and formulations for treating nail disorders. The system comprises an active agent formulation and a first barrier film. The active agent formulation includes an active agent and an aqueous liquid component. The first barrier film is configured to form a sheath over at least a portion of a finger or toe on which the nail is located. An optional second barrier film, with lower moisture vapor transmission rate than the first barrier film, is placed between the first barrier film and the active agent formulation (or between the active agent formulation and the external environment) to reduce the water evaporation from the active agent formulation. The sheath is configured to be capable of securing and retaining the active agent formulation within the sheath on a finger nail or toe nail while reducing evaporation of the liquid component of the aqueous liquid component of the active agent formulation. The active agent formulation can be a “slow-molding” hydrogel formulation that can slowly flow into the exact shape of the diseased nail to assure intimate contact and continuous delivery of the drug, while cannot be squeezed out of the position during the application.

### RELATED APPLICATION

This application claims the benefit of U.S. Provisional Application Ser. No. 61/010,500, filed Jan. 8, 2008.

### BACKGROUND OF THE INVENTION

Nail disorders, such as fungal infections or psoriasis of the nail, afflict millions of patients. Current therapies include oral and topical medications. Oral medications, such as the Lamisil® tablet, can be effective in many patients, but they have the potential to cause severe adverse side effects. Topical formulations are often in the form of lacquers (e.g. Penlac®) and generally have fewer adverse side effects. A typical lacquer formulation contains an active agent, a volatile solvent, and a lacquer-forming material such as poly[methylvinyl ether/maleic acid]. However, the efficacy of lacquer formulations is very low which is believed to be related to that fact that the volatile solvent evaporates quickly after the application, leaving the formulation without an excipient vehicle for sustained delivery of the active drug. As such, there remains a need for a treatment for these disorders which has few side effects and which is more effective than the use of lacquers.

### SUMMARY OF THE INVENTION

The present invention is drawn to systems and methods for treating nail disorders. In one embodiment, a system for treating a nail disorder can comprise an active agent formulation including an active agent for treating nail fungal infection or nail psoriasis and at least 10% water by weight, and a first barrier film. The first barrier film can be configured to form a sheath over the nail and the active agent formulation, wherein when applied to a digit of a subject, the sheath retains the active agent formulation therein, thereby reducing evaporation of said water from the active agent formulation.

In another embodiment, a method for treating nail disorder can comprise applying to a nail surface an active agent formulation comprising water and a drug selected from the group of antifungal agents and corticosteroids; and securing the active agent formulation to the nail surface with a first barrier film. The first barrier film can be configured to regulate evaporation of the water in the active agent formulation so that the water lost from the active agent formulation over a 12 hour period is no greater than 50%.

In another embodiment, a formulation for treating nail disorders can comprise an anti-fungal agent, a polymer capable of rendering the formulation slow molding, and water. This formulation can have a pH of from about 5 to about 8 and can have an osmolarity range with a lower limit which is isosmotic with 0.1% NaCl in water solution and an upper limit which is isosmotic with 3% NaCl in water.

### BRIEF DESCRIPTION OF DRAWINGS

FIG. 1 is a schematic bottom view of an unapplied, three-arm foldable first barrier film in accordance with embodiments of the present invention.

FIGS. 2A, 2B, and 2C depict three alternative schematic cutaway side views of the unapplied, three-armed foldable embodiment of FIG. 1, one with a tray or protective cover film (FIG. 2A), one without (FIG. 2B), and one with a tray or protective cover film as well as a second barrier layer between drug and the first barrier film (FIG. 2C).

FIG. 3 is a schematic side view of a sheath or tube first barrier film embodiment as it would appear when partially applied to a digit over a target nail.

FIG. 4 is a schematic bottom view of an unapplied foldable system with a first barrier film in accordance with embodiments of the present invention.

### DETAILED DESCRIPTION OF THE PREFERRED EMBODIMENT(S)

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “an active agent” includes reference to one or more of such active agents.

The term “drug(s)” or “active agent” refers to any bioactive agent that is applied to, into, or through the nail or surrounding skin which is applied for achieving a therapeutic affect, namely for treating nail disorders. This includes compositions that are traditionally identified as drugs, as well other bioactive agents that are not always considered to be “drugs” in the classic sense. When referring generally to a “drug,” or “active agent” it is understood that there are various forms of a given drug or active agent, and those various forms are expressly included. In accordance with this, various drug forms include polymorphs, salts, hydrates, solvates, and co-crystals.

The term “target nail” or “target surface” refers to a finger or toe nail which is afflicted with a disorder, e.g. psoriasis or a fungal infection, as well as the immediately surrounding skin area. Accordingly, the term “digit” of the target nail refers to a finger or toe on which the afflicted nail is present.

The term “slow molding” as used with respect to the active agent formulations of the present invention refers to formulations which are solid or semi-solid in appearance, but which are flowable and can change shape overtime, such as when acted upon by gravity or through long term applied pressure. A gel is said to be “slow molding” if it takes no less than 5 minutes but no more than 12 hours (the “molding time”) for 10 g of the gel, placed in a 20 mL vial (approximate dimensions 6 cm tall by 2.7 cm in diameter) with all air bubbles having been removed and the formulation is settled to the bottom of the vial, to flow flat (parallel with the long axis of the vial) when the vial is turned 900 from its upright position. Preferred slow molding gel embodiments can accomplish the above test protocol in no less than 15 minutes but no more than 6 hours.

One advantage of using a slow-molding formulation in the systems and methods of the present invention is that a slow-molding formulation can eventually flow into the exact shape of the target skin and nail surfaces and have intimate contact with those surfaces, while it cannot be squeezed out of the position during the application as a regular flowable liquid would. As the surface of a diseased nail is often quite coarse, a formulation that can flow to the shape of the nail surface can be important to insure intimate contact between the formulation and the nail surface and successful delivery of the active agent.

Further preferred slow molding gel embodiments can accomplish the above test protocol in no less than 30 minutes but no more than 4 hours. Detailed experimental parameters for the above described test for slow molding are set forth in Example 8.

As used herein, a “slow molding hydrogel” is a solid with the above slow-molding properties and contains at least about 40% water, often at least about 60% water, and more often at least about 80% water.

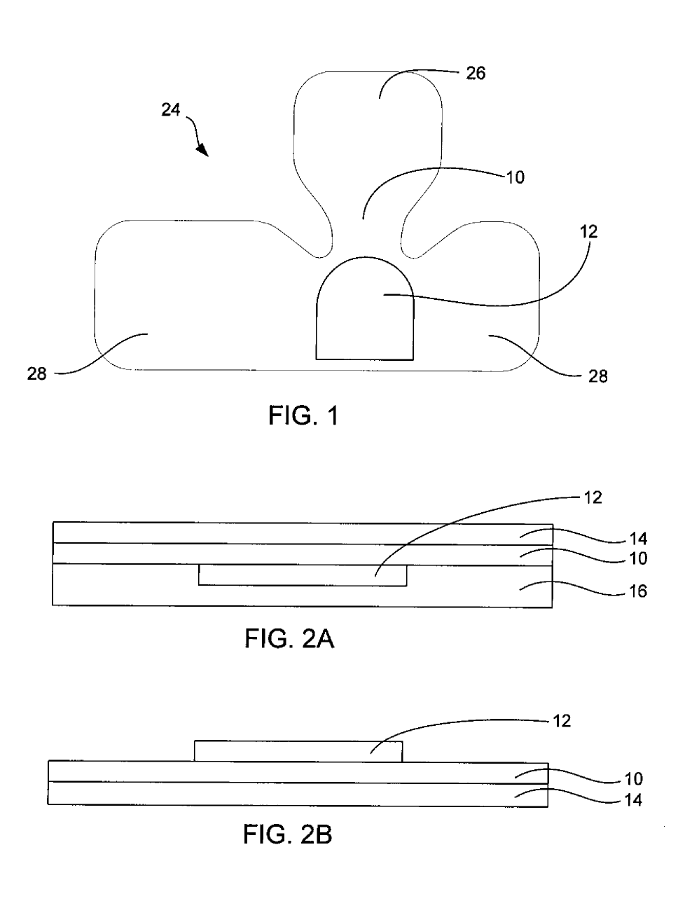

As used herein, the term “three-armed” when used in connection with the foldable first barrier film apparatuses and systems of the present invention refers generally to the configuration of the first barrier film which has three “arms,” e.g., one central arm and two lateral or adjacent arms, which collectively can be used to form a enclosed chamber or sheath when applied over the nail of a subject. Examples of “three-armed” embodiments are shown in FIGS. 1 and 4, although such embodiments are in no way intended to limit the possible design variations for such apparatuses. As shown in the figures, the arms do not need to be equal in proportion so long as they collectively are capable of forming the chamber or sheath discussed above.

As used herein, a plurality of drugs, compounds, and/or solvents may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0 mm” should be interpreted to include not only the explicitly recited values of about 0.01 mm to about 2.0 mm, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. This same principle applies to ranges reciting only one numerical value. Furthermore, such an interpretation should apply regardless of the breadth of the range or the characteristics being described.

With these definitions in mind, a system for treating nail disorders is provided which can comprise an active agent formulation and a first barrier film. The active agent formulation includes an active agent and an aqueous liquid component. The first barrier film is configured to form a sheath over at least a portion of a finger or toe on which the nail is located. The sheath can be configured to be capable of securing the active agent formulation on a finger nail or toe nail (retaining the formulation therein and in contact with the nail) while reducing evaporation of the liquid component of the aqueous liquid component of the active agent formulation.

In another embodiment, a system for treating nail disorders includes an active agent formulation and a first barrier film which is configured to secure and isolate the active agent formulation on a finger nail or toe nail while reducing evaporation of the liquid component of the aqueous liquid component of the active agent formulation. The active agent formulation includes an active agent and an aqueous liquid component and can be in the form of a hydrogel.

In another embodiment, a system for treating nail disorders includes an active agent formulation and a first barrier film which is configured to secure and isolate the active agent formulation on a finger nail or toe nail while reducing evaporation of the liquid component of the aqueous liquid component of the active agent formulation, as well as a second barrier film placed between the active agent formulation and the first barrier film (or between the active agent formulation and the external environment while the system is applied to the nail) to prevent the direct evaporation or permeation of liquid component(s) from the formulation through the first barrier film. The active agent formulation includes an active agent and an aqueous liquid component and can be in the form of a hydrogel.

In another embodiment, a method for treating nail disorders includes applying an active agent formulation which includes an aqueous liquid component to a nail surface and then securing and isolating the active agent formulation to the nail surface with a first barrier film which is configured to reduce evaporation of the liquid component of the aqueous liquid component of the active agent formulation.

The systems of the present invention can be configured for use in accordance with the methods disclosed herein, including configurations in which the system, including the active agent formulation and the first barrier film, can be left on the nail surface for extended period of times. In one embodiment, the system, after being appropriately applied, can be kept on the nail surface for a period of at least about 6 hours. In another embodiment, it can be kept on the nail surface for a period of at least about 20 hours after being appropriately applied. In another embodiment, the formulation and the first barrier film can be kept on the nail surface for a period of at least about 3 days after being appropriately applied. In yet another embodiment, the formulation and the first barrier film can be kept on the nail surface for a period of at least about 7 days after being appropriately applied. Thus, the first barrier film can be configured such that it, optionally with the help of the second barrier film, retains the formulation on the nail over the entire period of application time, e.g., at least 6 hours, 20 hours, 3 days, 6 days, or 7 days.

The system and methods of the present invention can also be effectively used to treat a wide range of nail disorders, including but not limited to fungal infections and psoriasis of the nail. Accordingly, the active ingredient or drug used in the system of the present invention can be chosen so as to be suitable to treat the targeted nail disorder. Some disorders can be targeted using the same active agents. When the disorder is a fungal infection, antifungal agents can be used. Non-limiting examples of anti-fungal agents which can be used include allylamines such as amorolfine, butenafine, naftifine, terbinafine, imidazoles such as fluconazole, itraconazole, ketoconazole, posaconazole, ravuconazole, voriconazole, clotrimazole, butoconazole, econazole, miconazole, oxiconazole, sulconazole, terconazole, tioconazole, bifonazole, others such as caspofungin, micafungin, anidulafingin, amphotericin B, AmB, nystatin, pimaricin, griseofulvin, ciclopirox olamine, haloprogin, tolnaftate, undecylenate, and combinations thereof. In one embodiment, the active agent can be terbinafine or a salt thereof. In another embodiment, the terbinafine is present in the active agent formulation in a concentration of about 0.1% to about 10%, and preferably from about 0.5% to about 5% by weight. In another embodiment, the active agent can be itraconazole. Generally, the concentration of the active agent used in the systems of the present invention can vary according to the nature and potency of the active agent. Determination of therapeutically appropriate amounts is within the skill of one of ordinary skill in the art. In one embodiment, the active agent can be present in the active agent formulation in both dissolved and undissolved form, thus providing sustained release delivery as the dissolved active agent is delivered and the undissolved active agent goes into solution over time.

When the nail disorder to be treated is psoriasis, the active agent can be one of the drugs described above, or alternatively, the drug can be a steroid. Non-limiting examples of steroids which can be used in the present invention include betamethasone dipropionate, clobetasol propionate, halobetasol propionate, diflorasone diacetate, amcinonide, desoximethasone, fluocinonide, halcinonide, mometasone furoate, betamethasone valerate, fluocinonide, fluticasone propionate, triamcinolone acetonide, fluocinolone acetonide, flurandrenolide, desonide, hydrocortisone butyrate, hydrocortisone valerate, alclometasone dipropionate, flumethasone pivolate, hydrocortisone, hydrocortisone acetate, tacrolimus, picrolimus, tazarotene, isotretinoin, cyclosporin, anthralin, vitamin D3, cholecalciferol, calcitriol, calcipotriol, tacalcitol, calcipotriene, and combinations thereof. In one embodiment, the steroid can be clobetasol or a derivative of clobetasol. In yet another embodiment, the steroid can be hydrocortisone. It is noteworthy that nail psoriasis may also be treated with the active agents set forth above as antifungal agents.

The active agent formulations used in the systems of the present invention contain both the active agent and a liquid component, and the liquid component can contain water (aqueous liquid component). The aqueous liquid component of the active agent formulations can include numerous solvents or other liquid components, so long as water is present in the composition, e.g., at least about 10 wt % of the liquid component in some embodiments. It is believed that in order to effectively treat nail disorders, particularly psoriasis or nail fungal infections, it is very helpful to hydrate the nail and keep it hydrated over an extended period of time in which the active agent is delivered. It is believed that hydration of the nail allows for adequate permeation of the active agent into and/or through the nail to treat the disorder. If a nail is not hydrated during the period of treatment, the active agent may not adequately permeate the nail; ergo, the active agent may not effectively treat the disorder. Hence, the inclusion of water in the active agent formulations of the present invention can be significant.

In one aspect of the present invention, water can comprise at least about 10 wt % of the active agent formulation. In another aspect, water can comprise at least about 50 wt % of the active agent formulation. In yet another aspect, water can comprise at least about 80% of the active agent formulation. Further, the liquid component of the active agent formulation can be only water.

In order to help maintain water on the nail surface, the active agent formulation can include a water retaining agent. Non-limiting examples of water retaining agents which can be used in the present invention include glycerin, urea, propylene glycol, and combinations thereof.

In one embodiment of the invention, the liquid component of the active agent formulation can include, in addition to water, a liquid that is less volatile than water. The inclusion of a liquid which is less volatile than water can aid in maintaining the fluidity and/or suppleness of the active agent formulation, particularly when the active agent formulation is maintained on a nail surface for extended periods of time.

In one embodiment of the invention, the liquid component of the active agent formulation can include, in addition to water, an ingredient that is believed to be able to reduce skin irritation. Non-limiting examples of ingredient that is believed to be able to reduce skin irritation include glycerin, honey, vitamin E, allantion, propylene glycol, and combinations thereof.

In another embodiment of the invention, the active agent formulation comprises undissolved active agent. As the dissolved active agent permeates into the nail and/or surrounding skin surfaces, the undissolved active agent dissolves into the solution and maintains relatively constant permeation driving force.

As the active agent formulations of the present inventions may contact the skin which surrounds the treated nail, the active agent formulations can be formulated to have an osmolarity which is within a “skin-friendly” osmolarity range. The “skin friendly” osmolarity range is defined as having a lower limit which is isosmotic with 0.1 wt % NaCl in water solution and as having an upper limit which is isosmotic with 3 wt % NaCl in water. In one embodiment, the formulations of the present invention can be formulated to have an osmolarity within an osmolarity range having a lower limit which is isosmotic with 0.4 wt % NaCl in water solution and having upper limit which is isosmotic with 1.5 wt % NaCl in water solution.

In another aspect of the present invention, the formulations of the present invention can have an osmolarity that is within 20% of that of normal human interstitial fluid. In yet a further aspect, the active agent formulation can have an ionic strength that is within 20% of that of normal human interstitial fluid. In yet another aspect, the active agent formulation can be formulated to have a pH of from about 5 to about 8.

When applied to the nail affected by the disorder, the active agent formulation can be applied in a layer having a thickness from about 0.5 millimeter to about 20 millimeters. In one embodiment, the active agent can be applied as a layer of at least about 1 millimeter. In another embodiment, the active agent formulation can be applied as a layer of at least about 2 millimeters. In yet another embodiment, the active agent formulation can be applied as a layer having a thickness of at least about 4 millimeters. In an additional embodiment, the active agent formulation can be applied as a layer having a thickness of at least about 6 millimeters.

In order to aid with the effective delivery of the active agent from the active agent formulation, the active agent formulation can be formulated to be viscous so it does not readily run off or is not readily displaced from the nail surface. In one embodiment, the active agent formulation can have a viscosity of at least about 500,000 centipoises. Alternatively or additionally, in order to maintain a highly viscous active agent formulation, in one embodiment, the active agent formulation can include a thickening agent. Generally, any thickening agent known in the pharmaceutical arts can be used so long as it does not interfere with the active agents of the formulation. Non-limiting examples of thickening agents include poly acrylic polymers, cellulose derivatives, gelatin and combinations thereof.

In another embodiment, the active agent formulation can be slow-molding. As defined above, slow-molding formulations are formulations which are solid or semi-solid in appearance, but which are flowable and can change shape over relatively long periods of time (a minute or so to hours) under the effect of gravity or pressure. In one embodiment of the present invention, the slow-molding active agent formulation is present in the system in the form of a hydrogel. The active agent formulation can be made as a hydrogel by including at least one hydrogel forming agent in the active agent formulation.

Any pharmaceutically acceptable hydrogel forming agents or agent combinations can be used so long as they do not interfere with the delivery of the active agent. Example of hydrogel forming agents or agent combinations include, but are not limited to polyvinyl alcohol, cellulose derivatives such as hydroxyl propyl cellulose, polyvinylpyrrolidone, carrageenin, pectin, polyacrylic acid, gelatin, hydrophilic polymerizable monomer (e.g., acrylamide) and combinations thereof. Preferred hydrogel forming agents or agent combinations can include polyvinyl alcohol with any of boric acid, sodium borate, and/or maleic anhydride containing polymers (Gantrez) as a crosslinking agent; polyacrylic acid with gelatin and/or chitosan as crosslinking agent, or acrylamide with bis(acryloylcystamine) as a crosslinking agent. In one embodiment, the hydrogel can have thermo-reversible properties. Thermo-reversible materials are well known in the art. Non-limiting examples of thermo-reversible materials which can be used in the systems and methods of the present invention can include one or more Pluronic® polymer distributed by BASF. In one aspect of the invention, the thermo-reversible material can have a melting point of from 10° C. to 34° C. In another embodiment of the invention, the active agent formulation hydrogel can be in the form of a hydrogel patch.

One method of maintaining the active agent formulation in appropriate contact with the target nail is by presenting the active agent formulation in a spongy material, or sponge, capable of retaining the active agent formulation. The active agent formulation should have a high enough viscosity such that it cannot be readily squeezed out of the sponge when a pressure of 20 g/cm² is applied for a short period of time, such as 5-10 seconds. In one embodiment, the spongy material can be open-celled so that it can hold a large volume of the formulation. A slow-molding hydrogel typically has sufficiently high viscosity to meet this standard. Use of the spongy material in the systems and methods of the present invention facilitates the continuous hydration of the nail during the treatment period. The spongy material also allows for the viscous or slow-molding active agent formulation to mold to and penetrate into the rough and coarse surface of the nail. While the slow-molding hydrogel formulation and the use of the spongy material both can independently facilitate maintaining the active agent formulation on the targeted nail and skin area, the combination can be even more effective. The use of the spongy material allows a thicker layer of the active agent formulation to be placed on the surface of the diseased nail and/or surrounding skin area, thus the formulation can be used for longer periods of time since it loses its liquid component at slower rates and higher quantities of the drug can be placed in the system.

Generally, any sponge-like material can be used, but hydrophilic, open-celled spongy materials are particularly useful. The sponge can be formed in any shape, but generally has at least one facial surface which has a surface area sufficient to cover a human nail. As human nails, both finger nails and toe nails, can vary in size depending on the person and digit of the nail, it is understood that a variety of sizes of spongy material can be used, so long as the surface area of at least one of its faces is sufficiently large to substantially cover the targeted nail surface. In another embodiment, the surface area of at least one of the faces of the spongy material is sufficiently large to substantially cover the targeted nail surface and most of the skin area within about 2 millimeter from the nail. In another embodiment, the surface area of at least one of the faces of the spongy material is sufficiently large to substantially cover the targeted nail surface and most of the skin area within 5 millimeter from the nail. In one embodiment, the spongy material can have a thickness of about 0.2 to 20 millimeters. In another embodiment, the spongy material can have a thickness of about 2 to 10 millimeters. In yet another embodiment, the spongy material can have a thickness of at least about 4 millimeters.

In one embodiment of the present invention, the active agent formulation can be present in the spongy material. The present invention provides for a method of loading a hydrogel formulation into an absorbent material, such as a sponge. The method includes the steps of adding a solution comprising a crosslinkable but substantially uncrosslinked polymer (hydrogel forming agent) to an absorbent material so that the solution has sufficiently low viscosity to be absorbed into the absorbent or sponge material. After the hydrogel forming solution is added to the absorbent material, a solution containing a cross-linking agent (i.e. 0.5 wt % to 10 wt %) can be added to the absorbent material so that the polymer and the cross-linking agent contact each other to form a cross-linked hydrogel within the absorbent or spongy material. The cross-linking agent used in the formation of the hydrogel can be any pharmaceutically acceptable cross-linking agent known in the art, including but not limited to boric acid, sodium borate, Gantrez (ISP), gelatin, and combinations thereof. In one embodiment, the hydrogel forming agent can be polyvinyl alcohol and the cross-linking agent can be boric acid or sodium borate. In such a method, the active agent can be present in either or both of the solution comprising the uncrosslinked polymer and in the cross-lining agent solution, before the solutions are combined.

Whether the active agent formulation is in the form a viscous fluid or a slow-molding hydrogel, it can be secured and isolated on a nail using a barrier film (referred as the first barrier film, to be distinguished from the second barrier film which will be described later). The first barrier film forms a seal or sheath around the nail, and in some cases, around the entire tip of the associated digit, which occludes the active agent formulation from the external environment, thereby reducing the evaporation of the active agent formulation, particularly the water that is included therein. Although evaporation from the active agent formulation is reduced by the first barrier film, it is preferable that the first barrier film be breathable to moisture so as avoid skin irritation when the system of the present invention is applied for extended periods of time, e.g. 6 hours to 7 days. In one embodiment, the first barrier film can have a moisture vapor transfer rate (MVTR) from about 50 g/m²/24 hr to 10,000 g/m²/24 hr. In another embodiment, the first barrier film can have the moisture vapor transfer rate (MVTR) from about 100 g/m²/24 hr to about 2,000 g/m²/24 hr. It should be noted that although the first barrier film can have the aforementioned MVTR, it is not an absolute requirement for the current invention. The first barrier film can have an MVTR outside the aforementioned range and still perform one of its most important functions: keeping the formulation on the targeted nail and skin surfaces.

In one aspect of the invention, the first barrier film can act as a barrier to bulk fluid. Thus, a typical bandage, such as a Band-Aid brand bandage, which is designed to more readily breath, often will not provide the barrier layer that is desired to keep the nail hydrated for extended periods of time in accordance with embodiments of the present invention. In other words, breathability of the first barrier film can be useful to provide some evaporation in order to reduce skin irritation and provide other benefits, but the evaporative properties of the system should retain moisture on the nail for extended periods of time more so than a typical bandage dressing.

The first barrier films of the present invention can be made a variety of materials, so long as the material provides ample occlusion and evaporation reduction. Non-limiting examples of first barrier films which can be used include polyurethane, polyethylene, polyester, and combinations or blends thereof. In one embodiment, the first barrier film can be a polyurethane film. The first barrier film can also be made of a material with lower than preferred MVTR, but with appropriate size and density of small perforated holes to increased the MVTR to a desired point.

As discussed above, the first barrier film forms a seal or sheath around the nail or digit tip, thereby securing and isolating the active agent formulation. In one embodiment, the first barrier film forms a sheath which encloses the nail and the skin within about 4 mm from the nail. In another embodiment, the first barrier film forms a sheath which encloses the nail and the skin within about 5 mm from the nail. In another embodiment, the first barrier film forms a sheath which encloses the nail and the skin within about 8 mm from the nail. In another embodiment, the first barrier film forms a sheath around the entire tip of the digit of the targeted nail, or even the entire digit.

The first barrier film can be configured in a variety of ways so as to form the seal or sheath around the nail. FIG. 1 shows an unapplied, three-arm foldable barrier system 24 for use in treating a nail disorder. The active agent formulation is located in a sponge 12, which has the shape similar to a nail but slightly larger. The sponge is adhered to the first barrier film 10. The first barrier film has three extended arms: two lateral arms 28 and a frontal arm 26, which can independently fold around the digit (not shown). For example, the frontal arm 26 can be folded down and over a tip of the finger or toe to which it is applied, thereby forming the frontal portion of the seal or sheath. The two lateral arms 28 can wrap the digit to which they are applied, thereby forming side and base portions of the seal or sheath. The arms of the three-armed, foldable barrier system can be, but do not need to be, of sufficient size to form a complete sheath around the finger or toe to which it is they are applied. In one embodiment, the lateral arms and the frontal arm become connected on a back side of the digit, sealing the entire digit within the sheath, though this is not required.

The sheath formed by the arms of the three-armed, foldable barrier system can substantially to completely seal the active agent formulation within the sheath so as to prevent direct exposure to the external environment and to prevent leaks of the active agent formulation. The sheath can act as a protective barrier against the entry of water and other fluids from the external sources in the environment, such as water that contacts the sheath when a subject bathes. The ability of the sheath the prevent leaks of the active agent formulation as well as protect against entry of external fluids enhances patient compliance as well as the overall effectiveness of the system in treating the nail disorder. Specifically, the ability of the sheath to protect against entry of fluids from the external environment allows the system to be used over an extended period of time without impeding the patient's ability to continue with normal everyday activities.

Another configuration of the first barrier film system of the present invention is shown in FIG. 4. FIG. 4 depicts an unapplied foldable barrier system 25 for use in treating a nail disorder. The system has similar components as the three-arm foldable system of FIG. 1 including an active agent containing sponge 12, a first barrier film 10 having two lateral arms 28 and a frontal arm 26. In the embodiment shown in FIG. 4, the lateral arms and the frontal arm are partially separated by two slits or perforated regions 18 that allow the arms to independently fold around the digit (not shown).

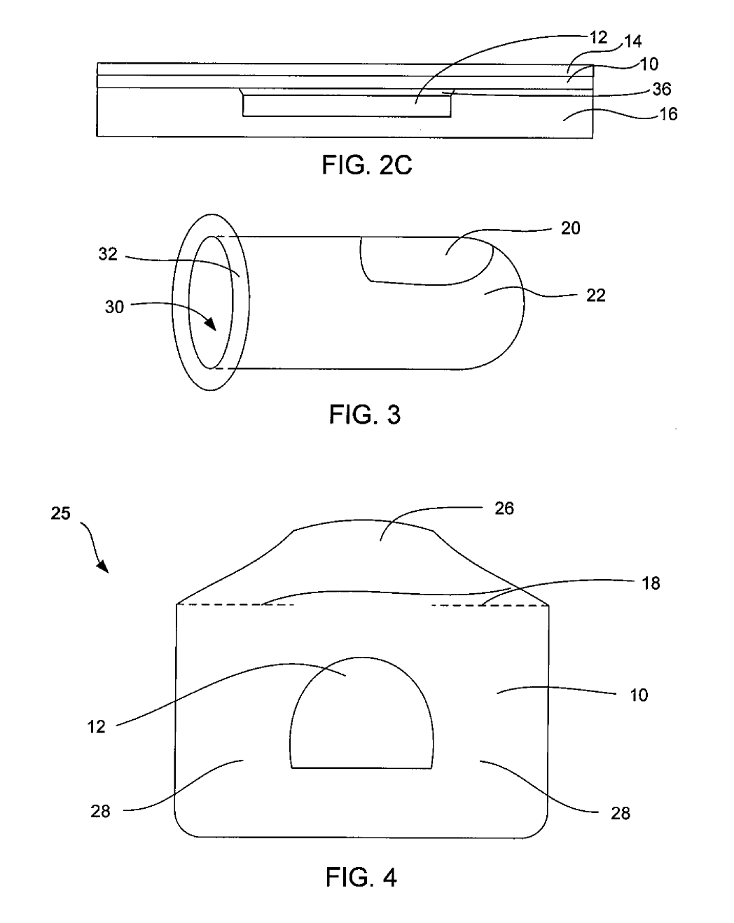

The first barrier film can be coated with a layer of adhesive to form a first barrier tape. For purposes of the present invention, the phases “first barrier film” and “first barrier tape” can be used interchangeably. The presence of an adhesive on the first barrier film allows the first barrier film to adhere to the skin of the finger or toe of the treated nail. The adhesive is generally located on at least the side of the first barrier film on which the sponge or sponge-like material is located. Once the system is applied, the adhesive prevents the first barrier film from sliding or rotating. FIG. 2A and FIG. 2B depict side perspectives of both a packaged (2A) and unpackaged/unapplied (2B) three-armed, foldable barrier system of the present invention. As shown in FIG. 2A, the first barrier film 10 and sponge 12 are packaged between an optional removable backing 14 and a release liner 16 of some type. In one embodiment, the release liner is nothing more than a protective film that is removed prior to application. However, in another embodiment, the release liner is actually a tray that is rigid, accepting the sponge in a pocket or defined housing, thereby retaining the integrity of the shape of the sponge and/or hydrogel which contains the water and the active agent. In other words, as a packaging tray, this structure can have an indentation which is configured to hold the sponge and the active agent formulation and protect them from the external environment prior to use. The tray also prevents evaporation of the liquid component of the active agent formulation during storage. The tray or release liner, as well as the removable backing, can be removed from the first barrier film/sponge prior to their application to a target nail. FIG. 2B, on the other hand, shows a side perspective view of the removable backing film 14, first barrier film 10, and the sponge 12, after removal from the tray. It is noted that the optional backing film is used to retain some rigidity to the first barrier film so that the user can apply relatively thin and very malleable membranes to the skin with ease.

In another embodiment, the first barrier film can be in the form of a sheath or tube which can be rolled onto or pulled over the digit of the target nail, see FIG. 3. A sponge 20 containing the active agent formulation can be attached to interior portion of the first barrier film sheath 22 in manners such that it resides over the nail of the finger or toe when the first barrier film sheath is rolled or pulled over the digit. The lower portion of the first barrier film 30 can have an adhesive applied thereto so as to create a seal around the base of the first barrier film and to prevent the film from rotating around or falling off of the digit. In some situations it may be desirable to coat all or a substantial portion of the interior of the first barrier film sheath with an adhesive. In this embodiment, the sheath can be unrolled over the digit, as shown partially unrolled at 32. In this embodiment, both the nail to be treated and the end of the finger or toe that contains the nail to be treated are covered within the sheath.

In another embodiment, a second barrier film 36 is placed between the first barrier film and the active agent formulation (FIG. 2C), or between the active agent formulation and the external environment when the system is applied to the nail. As mentioned earlier, the moisture vapor transfer rate of the first barrier film preferably is in a desirable range to provide skin friendliness. The second barrier film has much lower moisture vapor transfer rate and is to prevent or significantly reduce the permeation of the liquid component in the formulation directly from the formulation to the external environment through the first barrier film. The moisture vapor transfer rate (MVTR) of the second barrier film is preferably below about 50 g/m²/24 hr, and most preferably below 20 g/m²/24 hr.

In yet another embodiment, a method for treating a nail disorder is provided. The method requires applying an active agent formulation comprising water and drug for treating the nail disorder to a nail surface. The active agent can be selected from an antifungal agent or a corticosteroid. The active agent formulation is secured and isolated to the nail surface with a first barrier film configured to reduce evaporation of the water in the active agent formulation such that the water loss over a 12 hour period of typical use is no more than 50%. In one embodiment, the active agent formulation can be maintained on the nail surface for at least about 24 hours. In another embodiment, the active agent formulation can be maintained on the nail surface for at least about 3 days. In yet another embodiment, the active agent formulation can be maintained on the nail surface for at least about 6 days.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

### Loading a Slow-Molding Formulation into a Sponge-Like Material

It is difficult to load slow-molding formulations directly into spongy materials because of their high viscosities and low flow properties. To overcome this, a poly vinyl alcohol (PVA, m.w. approximately 30,000-50,000) in water solution with 2 to wt 15% PVA is loaded into the spongy material as a low viscosity solution. After loading of the PVA solution into a flat piece of hydrophilic spongy material (about 3 mm thick, each square centimeter of the spongy material is loaded with about 0.25 gram of the PVA solution), a second solution containing 0.5 wt % to 10 wt % of a crosslinking agent such as boric acid, sodium borate, or the like, is then added to the spongy material (i.e. 0.1 mL of the boric acid solution to each square centimeter of the spongy material). The PVA solution and the crosslinking agent solution mix by diffusion and the crosslinking agent crosslinks the PVA to form a slow-molding hydrogel within the spongy material.

### Example 2

### Loading a Slow-Molding Formulation into a Sponge-Like Material

Same as Example 1, except the polymer solution includes an antifungal agent, terbinafine, in sufficient amounts to treat a nail fungal infection, e.g. 2% terbinafine by weight

### Example 3

### Loading a Slow-Molding Formulation into a Sponge-Like Material

Same as Example 1, except that the cross-linking solution includes sufficient steroid for treating nail psoriasis, e.g. 0.05% clobetasol.

### Example 4

### PVA Slow-Molding Formulation

A PVA hydrogel is made by mixing a solution of polyvinyl alcohol (MW 30,000-50,000) with a sodium borate solution to form a solution having 10 wt % PVA and 5% sodium borate.

### Example 5

### Skin Permeation Methodology

Human Epidermal Membrane (HEM) is used as the model membrane for the in vitro flux studies described herein. Dermatome skin is received from various skin banks and is stored in freezer at −20° C. Before starting an experiment, skin is thawed at room temperature until it reaches ambient temperature. Small rectangular pieces of skin membrane are cut and mounted carefully between the donor and receiver chambers of a Franz diffusion cell. The receiver chamber is filled with pH 5.2 phosphate buffered saline (PBS). The experiment is initiated by placing test formulations on the stratum corneum (SC) of the skin sample. Franz cells are placed in a heating block maintained at 37° C. and the HMS temperature is maintained at 35° C. At predetermined time intervals, 800 μL aliquots are withdrawn and replaced with fresh PBS solution. Skin flux (mcg/cm²/h) is determined from the steady-state slope of a plot of the cumulative amount of drug permeated through the skin versus time.

### Example 6

### In Vitro Terbinafine Flux Across HEM from Slow-Molding PVA Formulations

Flux of terbinafine across human cadaver skin from slow molding polyvinyl alcohol formulations (PVA) crosslinked with borate containing 1-4% terbinafine was studied in an in vitro system outlined in Example 5. A commercially available 1 wt % terbinafine cream (e.g. “Athlete's Foot Cream”, terbinafine hydrochloride cream 1%, distributed by Walgreen) was used as a control. The ingredients of each formulation are noted in Table 1 below. PVA was dissolved in water by placing the aqueous solution containing PVA in the oven for several hours. After the PVA was dissolved, terbinafine was added and mixed/vortexed for 5-10 minutes to get uniformly dispersed (/partially dissolved) PVA-terbinafine solution. Then borate was added to crosslink the polymer to the desired consistency.

TABLE 1
Ingredients in the PVA Terbinafine Formulations (% w/w)

| | |
|---|---|
| Ingredients | Formulation |

| (weight percent) | 1 | 2 | 3 | 4 |
|---|---|---|---|---|
| PVA | 7.3 | 7.3 | 7.3 | 7.2 |
| Water | 90.4 | 89.4 | 88.4 | 87.5 |
| Borate | 1.3 | 1.3 | 1.3 | 1.2 |
| Terbinafine HCl | 1 | 2 | 3 | 4 |

TABLE 2
In vitro terbinafine skin permeation

| | Skin Flux* |
|---|---|
| Formulation No. | (mcg/cm²/h) |
| 1 | 0.6 ± 0.1 |
| 2 | 0.63 ± 0.04 |
| 3 | 0.9 ± 0.1 |
| 4 | 0.6 ± 0.1 |
| Control cream 1% Terbinafine | 0.27 ± 0.02 |

*Skin flux measurements represent the mean and standard deviation of three determinations. Flux measurements reported were determined from the linear region of the cumulative amount versus time plots.

Steady state terbinafine flux values for formulations 1-4 are similar within the scatter of the data, or in other words there is no dependence of terbinafine flux values as a function of drug concentration. Formulations 1-4 have flux values 2-3 times higher than the control cream. This example demonstrates that the PVA based hydrogel formulations have higher terbinafine flux than the control cream.

### Example 7

### Terbinafine Uptake into Nail Clippings

Terbinafine uptake into nail clippings when exposed to a slow-molding PVA formulation was compared to the uptake of terbinafine into nail clipping samples when exposed to the same control cream used in Example 6. Known weights of about 0.1 g of nail clippings were submerged in about 4 g of PVA or control cream formulations. After 4.5 days the nail clippings were removed from the formulations, excess formulation was wiped off the nails using a Kimwipe and then placed in 5 mL of absolute ethanol. The nail-ethanol solution was stirred constant through out the experiment. At 5 minutes, 1 mL ethanol solution was taken out and replaced with fresh 1 mL of absolute ethanol. Thereafter, removal of 1 mL of the solution and replacing with 1 mL of ethanol at predetermined time points were carried out over a period of time. An aliquot of the ethanol was then assayed in each sample taken at different time for terbinafine content.

TABLE 3
Ingredients in the PVA Terbinafine Formulations (% w/w)
used in the nail uptake study

Formulation (w/w %)

| Ingredients | 1 | 5 |
|---|---|---|
| PVA | 7.3 | 7.2 |
| Water | 90.4 | 89.6 |
| Sodium Borate | 1.3 | 1.3 |
| NaCl | — | 0.9 |
| Terbinafine HCl | 1 | 1 |

The formulations noted in Table 3 were prepared in the same manner as outlined in Example 6. The NaCl in Formulation 5 was added to the PVA/water solution at the same time as the terbinafine. Terbinafine uptake per gram of nail for each of the formulations studied are summarized in Table 4.

TABLE 4
Equilibrium terbinafine uptake into nail clippings.

| | Formulation | | |
|---|---|---|---|
| | | | Control |
| Ingredients | 1 | 5 | cream |
| Terbinafine uptake | 3104 | 2389 | 921 |
| (mcg/g nail) | | | |

These results show that the PVA formulations have a higher uptake of terbinafine into the nail versus the control cream which may result in higher flux values of terbinafine across the nail plate because of the higher drug content in the nail.

### Example 8

### Experimental Method to Define Slow-Molding Formulation

The slow molding PVA formulations used in this example are summarized in Table 5 below.

TABLE 5
Slow molding PVA formulations.

| | Formulation (w/w %) | | | | |
|---|---|---|---|---|---|
| Ingredients | 6 | 7 | 8 | 9 | 10 |
| PVA | 3.7 | 7.4 | 7.4 | 7.36 | 14.8 |
| Water | 95.0 | 92.1 | 91.6 | 91.89 | 83.9 |
| Sodium | 1.3 | 0.5 | 1.0 | 0.74 | 1.3 |
| Borate | | | | | |

The PVA (placebo) formulations were prepared by adding the specified amount of PVA solution and borax solution. Initially a PVA stock solution was prepared by adding known amount of PVA and water in a glass jar with a screw cap lid. The PVA solution was placed in the oven until the PVA dissolved in the water. A known amount of PVA solution was taken and then placed in a 20 mL scintillation vial (approximate dimensions of 6 cm high (from base to top of vial with cap in place), and 2.7 cm in diameter). To this, a known amount of sodium borate solution (prepared separately) was added so that to have the concentration of PVA, borax and water as mentioned in the Table 5. After adding both PVA and borax solution, the solution was then mixed thoroughly, and then centrifuged to remove any air bubbles. The formulations were made in such way that all the scintillation vials have same amount (10 g) of the formulations No. 6, 7, and 8. To begin the experiment, the 20 mL vial with the formulation is placed in the upright position and the formulation in the vial is flat at the formulation head space interface with no residual formulation on the sides of the vial above this interface. Once the formulation is deemed level, the vial is then turned 900 so that the long axis of the vial is parallel with the countertop. Once the vial is turned a timer is started and the time for the PVA formulation to flow so that the formulation/headspace interface is parallel with the long axis of the vial is determined. The time needed for the formulation to flow to this equilibrium position is defined as the “molding time.”

TABLE 6
molding times for PVA formulations

| | Formulation | | | | |
|---|---|---|---|---|---|
| | 6 | 7 | 8 | 9 | 10 |
| molding | 3-5 | 30-40 | 300-330_{—} | 180-210 | >960 |
| time (min) | | | | | |

Formulation 6 flows parallel to the shape of the glass vial (parallel to the long axis of the vial after it is turned 90) in 3-5 minutes, Formulation 7 which has more PVA than Formulation 6 takes a longer amount of time to flow to the equilibrium position and Formulation 8, which has similar PVA but more borate than Formulation 7, takes up to 5 to 5.5 hours to flow parallel. These results indicate that by manipulating the concentrations of PVA and borate the molding time can be adjusted to a desired point or a narrow range. Such manipulations could be readily accomplished by one of ordinary skill in the art.

### Example 9

### Thiol-Containing Slow Molding Formulation

A hydrogel formulation showing slow molding characteristics is prepared from polymers containing thiol groups which are crosslinked through a thiol-disulfide crosslinking mechanism. This hydrogel is made by mixing the correct ratio of a hydrophilic polymerizable monomer (e.g., acrylamide) and bis(acryloylcystamine).

### Example 10

### Preparation of PVA Slow Molding Formulation by Freezing and Thawing Cycles

Hydrogel formulations showing slow molding characteristics are prepared by carrying out freezing and thawing of aqueous solution of poly(vinyl alcohol). Aqueous poly(vinyl alcohol) (PVA) solution is known to undergo (physical) crosslinking and provide crosslinked PVA gels when it is subjected to freezing (keeping at −5° C.) and thawing cycle repeatedly (keeping at ˜25° C.). The properties of the gel depends on polymer molecular weight, concentration of the PVA in solution, the temperature and time of the freezing and thawing, and the number of freeze-thaw cycles.

### Example 11

### In Vitro Terbinafine Permeation as a Function of pH

Permeation of terbinafine across human cadaver skin from slow molding polyvinyl alcohol formulations (PVA) crosslinked with sodium borate containing 1-4% terbinafine was studied in an in vitro system outlined in Example 5. The ingredients of each formulation are noted in the table below. PVA was dissolved in water by placing the aqueous solution containing PVA in the oven for several hours. After the PVA was dissolved, terbinafine was added and mixed/vortexed for 5-10 minutes to get uniformly dispersed (partially dissolved) PVA-terbinafine solution. Then borate was added to crosslink the polymer to the desired consistency.

TABLE 7
Ingredients in the PVA Terbinafine Formulations (% w/w)

| | Formulation (in wt %) | | | | |
|---|---|---|---|---|---|
| Ingredients | 11 | 12 | 13 | 14 | 15 |
| PVA | 7.3 | 7.3 | 7.3 | 7.3 | |
| Water | 89.4 | 89.4 | 89.4 | 89.4 | 92.8 |
| Borate | 1.3 | 1.3 | 1.3 | 1.3 | |
| Terbinafine HCl | 2.0 | 2.0 | 2.0 | 2.0 | 1.0 |
| Partially Neutralized Polyacrylate | — | — | — | — | 4.2 |
| Gelatin, Type-2 | — | — | — | — | 2.0 |
| pH | ~5 | ~6 | ~7 | ~8 | ~5.5 |

TABLE 8
In vitro terbinafine skin permeation

| | Skin Flux* |
|---|---|
| Formulation No. | (mcg/cm²/h) |
| 11 | 3.0 ± 0.3 |
| 12 | 1.7 ± 0.1 |
| 13 | 1.5 ± 0.1 |
| 14 | 1.18 ± 0.05 |
| 15 | 3.6 ± 0.4 |
| Control cream 1% Terbinafine | 0.36 ± 0.04 |

*Skin flux measurements represent the mean and standard deviation of three determinations. Flux measurements reported were determined from the linear region of the cumulative amount versus time plots.

Steady state terbinafine skin flux values for formulations 11-14 are dependent on formulation pH. The lower pH formulations had higher drug solubility and higher terbinafine flux. Formulation 15 has even higher flux than Formulation 11, even with a higher pH. All formulations have significantly higher flux than the control 1% terbinafine cream.

It is not surprising that the flux values from the present example differs from the terbinafine flux results in previous example (i.e., Example 6) because of the variability inherent in the experimental set up and the differences in skin samples used. Therefore in vitro permeation values reported in these examples should be evaluated independently.

### Example 12

### Slow Molding Formulation Example Using Polyacrylate

A polyacrylate hydrogel is made by mixing a solution of partially neutralized polyacrylate with a gelatin (type-2) in an aqueous solution to form a solution having 2-4.5 wt % polyacrylate and 1.5-2.0% gelatin and the balance of the formulation containing water.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A system for treating a nail disorder, comprising:
an active agent formulation including an active agent for treating nail fungal infection or nail psoriasis and at least 10% water by weight; and
a first barrier film configured to form a sheath over said nail and said active agent formulation, wherein when applied to a digit of a subject, said sheath retains the active agent formulation therein, thereby reducing evaporation of said water from the active agent formulation.

2. A system as in claim 1, wherein the active agent formulation contains at least 50% water by weight.

3. A system as in claim 1, wherein the nail disorder is a nail fungal infection.

4. A system as in claim 3, wherein the active agent is an anti-fungal agent selected from the group consisting of amorolfine, butenafine, naftifine, terbinafine, fluconazole, itraconazole, ketoconazole, posaconazole, ravuconazole, voriconazole, clotrimazole, butoconazole, econazole, miconazole, oxiconazole, sulconazole, terconazole, tioconazole, bifonazole, caspofungin, micafungin, anidulafingin, amphotericin B, AmB, nystatin, pimaricin, griseofulvin, ciclopirox olamine, haloprogin, tolnaftate, undecylenate, and combinations thereof.

5. A system as in claim 3, wherein the active agent is terbinafine or a salt thereof, in a concentration of about 0.02% to about 10% by weight.

6. A system as in claim 1, wherein the nail disorder is nail psoriasis.

7. A system as in claim 6, wherein the active agent is selected from the group consisting of betamethasone dipropionate, clobetasol propionate, halobetasol propionate, diflorasone diacetate, amcinonide, desoximethasone, fluocinonide, halcinonide, mometasone furoate, betamethasone valerate, fluocinonide, fluticasone propionate, triamcinolone acetonide, fluocinolone acetonide, flurandrenolide, desonide, hydrocortisone butyrate, hydrocortisone valerate, alclometasone dipropionate, flumethasone pivolate, hydrocortisone, hydrocortisone acetate, tacrolimus, picrolimus, tazarotene, isotretinoin, cyclosporin, anthralin, vitamin D3, cholecalciferol, calcitriol, calcipotriol, tacalcitol, calcipotriene, and combinations thereof.

8. A system as in claim 1, wherein the first barrier film has the moisture vapor transfer rate (MVTR) of from 50 g/m²/24 hr to about 10,000 g/m²/24 hr.

9. A system as in claim 8, wherein the system further comprises a second barrier film placed between the active agent formulation and the first barrier film or between the active agent formulation and the external environment when the system is applied, and which has a moisture vapor transfer rate (MVTR) below 50 g/m²/24 hr.

10. A system as in claim 1, wherein the system further comprises a second barrier film placed between the first barrier film and the active agent formulation, wherein the moisture vapor transfer rate (MVTR) of the second barrier film is below 20 g/m²/24 hr.

11. A system as in claim 1, wherein the system further comprises a second barrier film placed between the active agent formulation and the external environment, wherein the moisture vapor transfer rate (MVTR) of the second barrier film is below 20 g/m²/24 hr.

12. A system as in claim 1, wherein said sheath encloses at least the nail and the skin area within 2 mm from the nail.

13. A system as in claim 1, wherein the system further comprises a spongy material capable of holding said active agent formulation.

14. A system as in claim 13, wherein the spongy material includes a face having a surface area sufficient to cover a human nail.

15. A system as in claim 13, wherein the spongy material has a thickness of about 0.2 to 10 millimeters.

16. A system as in claim 1, wherein the active agent formulation is a hydrogel formulation.

17. A system as in claim 1, wherein the active agent formulation is a slow-molding formulation.

18. A system as in claim 1, wherein the active agent formulation has an osmolarity within an osmolarity range having a lower limit which is isosmotic with 0.1 wt % NaCl in water solution and having an upper limit which is isosmotic with 3 wt % NaCl in water solution.

19. A system as in claim 1, wherein the active agent formulation has an osmolarity within an osmolarity range having a lower limit which is isosmotic with 0.4 wt % NaCl in water solution and having an upper limit which is isosmotic with 1.5 wt % NaCl in water solution.

20. A system as in claim 1, wherein the active agent formulation has an osmolarity within an osmolarity range having a lower limit which is isosmotic with 0.1 wt % NaCl in water solution and having an upper limit which is isosmotic with 3 wt % NaCl in water, and has a pH from about 5 to about 8.

21. A system as in claim 1, wherein the active agent formulation comprises poly vinyl alcohol, terbinafine or a salt of terbinafine, and water.

22. A system as in claim 1, wherein the active agent formulation comprises undissolved active agent.

23. A system as in claim 1, wherein the first barrier film comprises polyurethane.

24. A system as in claim 1, wherein the first barrier film is configured to have a three arm shape prior to application to the nail surface.

25. A system as in claim 13, wherein the active agent formulation is in the form of a slow-molding hydrogel and is held in a spongy material.

26. A system as in claim 1, wherein the active agent formulation is secured over the nail such that, other than due to breathability of the first barrier film, no active agent leaks outside the sheath and no fluid enters the sheath from external sources.

27. A method for treating nail disorder, comprising:
applying to a nail surface an active agent formulation comprising water and a drug selected from the group of antifungal agents and corticosteroids;
securing said active agent formulation to said nail surface with a first barrier film configured to regulate evaporation of the water in the active agent formulation so that the water lost from the active agent formulation over a 12 hour period is no greater than 50%.

28. A method as in claim 27, wherein the active agent formulation and the first barrier film are kept on the nail surface for a period of at least 24 hours.

29. A method as in claim 27, wherein the active agent formulation and the first barrier film are kept on the nail surface for a period of at least 3 days.

30. A method as in claim 27, wherein the active agent formulation and the first barrier film are kept on the nail surface for a period of at least 6 days.

31. A formulation for treating nail disorders, comprising:
a) an anti-fungal agent;
b) a polymer capable of rendering the formulation slow molding; and
c) water;
wherein the formulation has a pH of from 5 to about 8 and an osmolarity within an osmolarity range having a lower limit which is isosmotic with 0.1 wt % NaCl in water solution and having an upper limit which is isosmotic with 3 wt % NaCl in water.

32. A formulation as in claim 31, wherein the formulation has an osmolarity within an osmolarity range having a lower limit which is isosmotic with 0.4 wt % NaCl in water solution and having an upper limit which is isosmotic with 1.5 wt % NaCl in water.
